(19) United States
(12) Patent Application Publication
(10) Pub. No.: US 2009/0171283 A1
(43) Pub. Date: Jul. 2, 2009

(54) METHOD OF BONDING A DILATION ELEMENT TO A SURFACE OF AN ANGIOPLASTY BALLOON

(75) Inventors: Darin G. Schaeffer, Bloomington, IN (US); Kimberly D. Roberts, Bloomfield, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/965,499

(22) Filed: Dec. 27, 2007

(51) Int. Cl. A61M 25/10

(52) U.S. Cl. 604/103.08

(57) ABSTRACT

A balloon catheter with dilation elements and method of fabricating thereof is provided that may be used to dilate and/or cut hardened regions of a body vessel. The balloon catheter is provided with dilation elements that extend along a surface of a balloon. One or both ends of the dilation element is inserted through corresponding apertures formed in the balloon neck. After inserting the one or both ends through the corresponding apertures, the ends are bonded into the material of the catheter shaft and the balloon.

### BACKGROUND

The present invention relates generally to medical devices and more particularly to balloon catheters used to dilate narrowed portions of a lumen.

Balloon catheters are widely used in the medical profession for various intraluminal procedures. One common procedure involving the use of a balloon catheter relates to angioplasty dilation of coronary or other arteries suffering from stenosis (i.e., a narrowing of the arterial lumen that restricts blood flow).

Although balloon catheters are used in many other procedures as well, coronary angioplasty using a balloon catheter has drawn particular attention from the medical community because of the growing number of people suffering from heart problems associated with stenosis. This has lead to an increased demand for medical procedures to treat such problems. The widespread frequency of heart problems may be due to a number of societal changes, including the tendency of people to exercise less while eating greater quantities of unhealthy foods, in conjunction with the fact that people generally now have longer life spans than previous generations. Angioplasty procedures have become a popular alternative for treating coronary stenosis because angioplasty procedures are considerably less invasive than other alternatives. For example, stenosis of the coronary arteries has traditionally been treated with bypass surgery. In general, bypass surgery involves splitting the chest bone to open the chest cavity and grafting a replacement vessel onto the heart to bypass the blocked, or stenosed, artery. However, coronary bypass surgery is a very invasive procedure that is risky and requires a long recovery time for the patient.

To address the increased need for coronary artery treatments, the medical community has turned to angioplasty procedures, in combination with stenting procedures, to avoid the problems associated with traditional bypass surgery. Typically, angioplasty procedures are performed using a balloon-tipped catheter that may or may not have a stent mounted on the balloon (also referred to as a stented catheter). The physician performs the angioplasty procedure by introducing the balloon catheter into a peripheral artery (commonly one of the leg arteries) and threading the catheter to the narrowed part of the coronary artery to be treated. During this stage, the balloon is uninflated and collapsed onto the shaft of the catheter in order to present a low profile which may be passed through the arterial lumens. Once the balloon is positioned at the narrowed part of the artery, the balloon is expanded by pumping a mixture of saline and contrast solution through the catheter to the balloon. As a result, the balloon presses against the inner wall of the artery to dilate it. If a stent is mounted on the balloon, the balloon inflation also serves to expand the stent and implant it within the artery. After the artery is dilated, the balloon is deflated so that it once again collapses onto the shaft of the catheter. The balloon-tipped catheter is then retracted from the arteries. If a stent is mounted on the balloon of the catheter, the stent is left permanently implanted in its expanded state at the desired location in the artery to provide a support structure that prevents the artery from collapsing back to its pre-dilated condition. On the other hand, if the balloon catheter is not adapted for delivery of a stent, either a balloon-expandable stent or a self-expandable stent may be implanted in the dilated region in a follow-up procedure. Although the treatment of stenosed coronary arteries is one common example where balloon catheters have been used, this is only one example of how balloon catheters may be used and many other uses are also possible.

One problem that may be encountered with conventional angioplasty techniques is the proper dilation of stenosed regions that are hardened and/or have become calcified. Stenosed regions may become hardened for a variety of reasons, such as the buildup of atherosclerotic plaque or other substances. Hardened regions of stenosis can be difficult to completely dilate using conventional balloons because hardened regions tend to resist the expansion pressures applied by conventional balloon catheters. Although the inventions described below may be useful in treating hardened regions of stenosis, the claimed inventions may also solve other problems as well.

### SUMMARY

Accordingly, a balloon catheter with a dilation element and method of fabricating thereof is provided in which an end of the dilation element is bonded into the distal neck portion of the balloon and the shaft.

The invention may include any of the following aspects in various combinations and may also include any other aspect described below in the written description or in the attached drawings.

A balloon catheter for dilation of a vessel wall, comprising a shaft having a distal end and a proximal end; a balloon mounted on the distal end of the shaft, the balloon comprising a distal neck portion, a proximal neck portion, wherein at least a length of an outer surface of the balloon comprises a working diameter adapted to dilate the vessel wall, the shaft comprising an inflation lumen extending therethrough in fluid communication with an interior region of the balloon, the balloon thereby being expandable between a deflated state and an inflated state; and a dilation element comprising a proximal end, a distal end, and a middle portion, the middle portion of the dilation element extending along the working diameter of the balloon, the distal end of the dilation element extending through a distal aperture of the balloon at the distal neck portion, the distal end of the dilation element being bonded into the distal neck portion of the balloon and the shaft.

The balloon catheter, wherein the proximal end of the dilation element extends through a proximal aperture of the balloon at the proximal neck portion, the proximal end of the dilation element being bonded into the proximal neck portion of the balloon and the shaft.

The balloon catheter, wherein the middle portion of the dilation element comprises a wire.

The balloon catheter, wherein at least one of the proximal end and the distal end of the dilation element comprises a roughened surface.

The balloon catheter, wherein the middle portion of the dilation element is rigid and unattached to the working diameter of the balloon.

The balloon catheter, wherein at least one of the distal end and the proximal end of the dilation element comprises a coil.

The balloon catheter, wherein the proximal end of the dilation element is heat bonded into the proximal neck portion and the shaft.

The balloon catheter, wherein the distal end of the dilation element being bonded to the distal neck portion of the balloon and the shaft has a length between about 1 mm and about 2 mm.

The balloon catheter, wherein the distal end of the dilation element is heat bonded to the distal neck portion of the balloon and the shaft.

The balloon catheter, wherein the dilation element extends substantially parallel to a longitudinal axis of the shaft.

The balloon catheter, wherein a plurality of the dilation elements are circumferentially disposed about the balloon.

The balloon catheter, wherein the proximal end of the dilation element extends through a proximal aperture of the balloon at the proximal neck portion, the proximal end of the dilation element being bonded to the proximal neck portion of the balloon and the shaft, wherein the middle portion of the dilation element comprises a wire, wherein at least one of the proximal end and the distal end of the dilation element comprises a roughened surface, wherein the middle portion of the dilation element is rigid and unattached to the working diameter of the balloon, wherein the proximal end of the dilation element is heat bonded into the proximal neck portion and the shaft, and wherein the distal end of the dilation element is heat bonded into the distal neck portion of the balloon and the shaft.

A method of bonding a dilation element to a balloon, comprising the steps of: (a) positioning a dilation element along an outer diameter of a balloon, a middle portion of the dilation element extending along the outer diameter; (b) forming a proximal aperture along a proximal neck of the balloon; (c) inserting a proximal end of the dilation element through a proximal aperture located at the proximal neck of the balloon, the proximal end of the dilation element disposed between an inner diameter of the balloon at the proximal neck and an outer diameter of a shaft; and (d) heat bonding the proximal end of the dilation element with the balloon and the shaft.

The method, further comprising the steps of: (e) forming a distal aperture along a distal neck of the balloon; (f) inserting a distal end of the dilation element through the distal aperture located at the distal neck of the balloon, the distal end of the dilation element disposed between an inner diameter of the distal neck and an outer diameter of the shaft; and (g)

heat bonding the distal end of the dilation element with the balloon and the shaft.

The method, further comprising surface treating the proximal end of the dilation element to increase mechanical adhesion of the dilation element before inserting the proximal end through the proximal aperture.

The method, wherein step (d) further comprises mounting a bonding sleeve over the proximal neck of the balloon and the dilation element.

The method, wherein step (d) further comprises applying a sufficient amount of heat and pressure for a predetermined time to form the heat bond.

The method, further comprising placing a mandrel within a lumen of the shaft to prevent collapse of the lumen during the heat bonding step.

The method, wherein the middle portion of the dilation element comprises a wire and the proximal end of the dilation element comprises a coil.

The method, wherein the middle portion of the dilation element comprises a wire and a distal end of the dilation element comprises a coil.

### BRIEF DESCRIPTION OF SEVERAL VIEWS OF THE DRAWINGS

The invention may be more fully understood by reading the following description in conjunction with the drawings, in which:

FIG. 1 is a longitudinal cross-sectional view of a balloon catheter with an inflated balloon having cutting wires bonded to the neck of the balloon and catheter shaft;

FIG. 2 shows a longitudinal cross-sectional view of the balloon catheter of FIG. 1 prior to the bonding of the cutting wires;

FIG. 3 shows a partial cross-sectional view of the balloon catheter with a mandrel through the wire guide lumen and a second mandrel through the inflation lumen;

FIG. 4 is an end cross-sectional end view of the balloon catheter showing the wire guide lumen and inflation lumen;

FIG. 5 shows one example of a cannula inserted through the wire guide and inflation lumens during the heat bonding process; and

FIG. 6 shows a longitudinal cross-sectional view of an alternative balloon catheter in which each of the free ends of the dilation elements are bonded to the material of the balloon neck and catheter shaft.

### DETAILED DESCRIPTION

The embodiments are described with reference to the drawings in which like elements are referred to by like numerals. The relationship and functioning of the various elements of the embodiments are better understood by the following detailed description. However, the embodiments as described below are by way of example only, and the invention is not limited to the embodiments illustrated in the drawings. It should also be understood that the drawings are not necessarily to scale and in certain instances details have been omitted, which are not necessary for an understanding of the embodiments, such as conventional details of fabrication and assembly

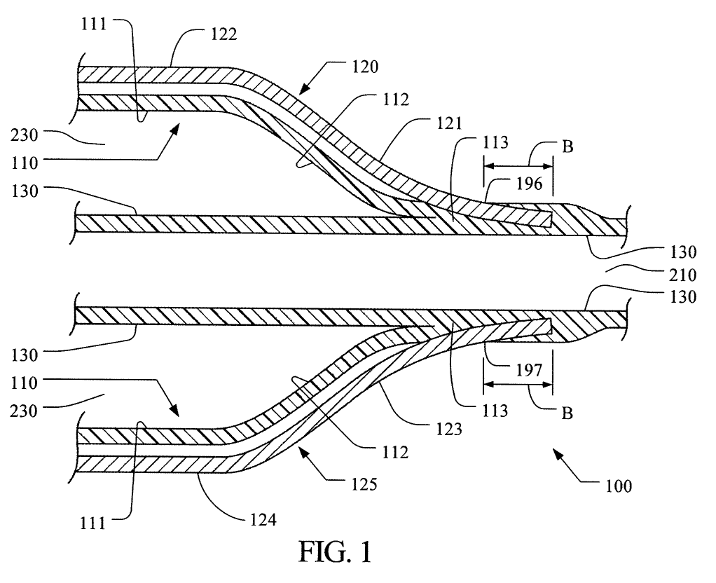

FIG. 1 shows an exemplary balloon catheter 100 after dilation elements 120 and 125 have been bonded into the surfaces of a balloon 110 and a catheter shaft 130. As used herein, the term “dilation element” refers to structural elements for dilating a vessel or cutting and/or rupturing hardened (e.g., calcified) lesions. In the example of FIG. 1, dilation elements 120 and 125 specifically refer to cutting wires 120 and 125. The balloon catheter 100 includes a shaft 130 and a balloon 110, which is shown in its inflated state.

Cutting wire 120 includes a proximal end, distal end 121, and middle portion 122. The middle portion 122 is defined as the portion of the cutting wire 120 that extends along the working diameter 111 of the balloon 110. The working diameter 111 extends along a part of the length of the balloon 110. Typically, the working diameter 111 of the balloon 110 is a portion that inflates to a generally uniform circumference in order to evenly dilate a section of a body vessel. However, the working diameter 111 does not necessarily need to have a uniform circumference. The working diameter 111 of the balloon 110 may be connected to the shaft 130 with a tapered proximal portion and a tapered distal portion 112. The length of the working diameter may be defined as the distance between the balloon proximal end, where the tapered proximal portion meets the working diameter 111, and the balloon distal end, where the tapered distal portion 112 meets the working diameter 111.

The distal end 121 of the cutting wire 120 is defined as the portion of the cutting wire 120 that extends along the tapered distal portion 112 of the balloon 110 and along the distal neck 113 of the balloon 110. A distal-most part of the distal end 121 may extend through distal aperture 196 of balloon 110 and thereafter be heat bonded to the balloon 110 and catheter shaft 130, as will be explained below.

The proximal end of the cutting wire 120 is defined as the portion of the cutting wire 120 that extends along the tapered proximal portion of the balloon 110 to the proximal neck of the balloon 110. A proximal-most part of the proximal end of cutting wire 120 may extend through a proximal aperture corresponding to distal aperture 196 and be heat bonded to the balloon 110 and catheter shaft 130, as will be explained below. The proximal aperture may longitudinally align with the distal aperture 196 of balloon 110.

Cutting wire 125 also includes a proximal end, distal end 123, and middle portion 124. Cutting wire 125 is shown disposed about 180° relative to cutting wire 120. The middle portion 124 is defined as the portion of the cutting wire 125 that extends along the working diameter 111 of the balloon 110. The distal end 123 is defined as the portion of the cutting wire 125 that extends along the tapered distal portion 112 of the balloon 110 and along the distal neck 113 of the balloon 110. A distal-most part of the distal end 123 may extend through a distal aperture 197 of balloon 110 and thereafter be heat bonded to the balloon 110 and catheter shaft 130, as will be explained below. The proximal end of the cutting wire 125 is defined as the portion of the cutting wire 125 that extends along the tapered proximal portion of the balloon 110 to the proximal neck of the balloon 110. A proximal-most part of the proximal end may be heat bonded to the balloon 110 and catheter shaft 130, as will be explained below.

As shown in FIG. 1, the distal-most part of the distal end 121 of cutting wire 120 and the distal-most part of the distal end 123 of cutting wire 125 are embedded into the surfaces of the balloon 110 and the catheter shaft 130 to form a bond. The bond is preferably a heat bond. Other types of bonds known to one of ordinary skill in the art are contemplated. Laser bonding, gluing, and chemical solvent bonding may be used. The regions of the bonds are defined by the designation “B”. The bonded regions are preferably the only points of attachment of the cutting wires 120 and 125 with the balloon 110. The middle portions 122 and 124 of cutting wires 120, 125 preferably remain unattached along the outer diameter of the balloon 110.

Although two cutting wires 120 and 125 are shown in FIG. 1, a single wire may be utilized. Alternatively, greater than two wires may be circumferentially disposed about the balloon catheter 100 and bonded to the balloon 110 and the catheter shaft 130. Preferably, each free end of the cutting wire is fed through an aperture of the balloon neck. Preferably, each aperture is dedicated to a single free end of a cutting wire. Alternatively, more than a single free end may be fed through an aperture of the balloon neck.

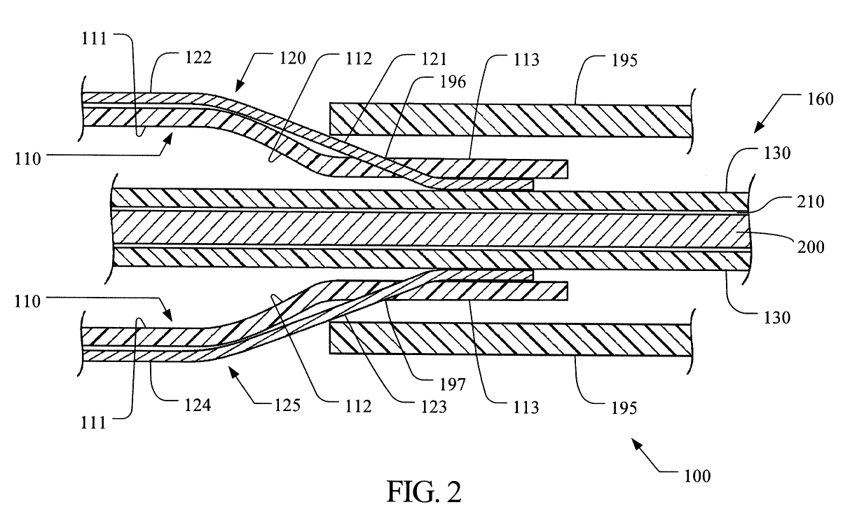

A method of connecting one or more dilation elements, such as cutting wires, to a surface of an angioplasty balloon will now be discussed with reference to FIG. 2. FIG. 2 shows a distal end 160 of the balloon catheter 100 before the cutting wires 120 and 125 have been heat bonded to the balloon 110. The balloon 110 is at least partially inflated to provide an unpleated surface onto which cutting wires 120 and 125 can be positioned. Distal aperture 196 is created along the distal neck 113 of the balloon 110. The distal aperture 196 may be sufficiently sized for the distal end 121 of cutting wire 120 to extend therethrough. A corresponding proximal aperture is preferably created along the proximal neck of the balloon 110. The proximal aperture may longitudinally align with the distal aperture 196. Similarly, distal aperture 197 is created along the distal neck 113 of the balloon 110. A corresponding proximal aperture to distal aperture 197 is preferably created along the proximal neck of the balloon 110. FIG. 2 shows that the distal aperture 197 is circumferentially disposed about 180° from distal aperture 196. Other angular separations between distal apertures 197 and 196 are contemplated.

The proximal apertures and distal apertures 196, 197 may be created by any means known to one of ordinary skill in the art, including hole punching the surface of the balloon 110. Alternatively, the apertures may be formed by utilizing the tip of the cutting wire 120, 125 to pierce through the proximal and distal neck 130 of the balloon 110. Preferably, the apertures may be formed by laser cutting.

Having created the distal apertures 196 and 197 along with their corresponding proximal apertures, cutting wires 120 and 125 may be positioned along the outer surface of the balloon 110. Specifically, the middle portion 122 of the cutting wire 120 is positioned along the working diameter 111 of balloon 110. Preferably, the middle portion 122 is aligned parallel to the longitudinal axis of the balloon catheter 100. The distal end 121 of the cutting wire 120 may then be positioned such that it extends along the tapered distal portion 112 of the balloon 110 and along the distal neck 113 of the balloon 110. The distal-most part of the distal end 121 may be fed through distal aperture 196. The length of the distal end 121 that is fed through distal aperture 196 may vary and is partly dependent upon the region that the balloon catheter 100 is to be deployed within. In the example shown in FIG. 2, the length of the distal end 121 that is fed through distal aperture 196 may range from about 1 mm to about 5 mm.

After the distal-most part of the distal end 121 is fed through distal aperture 196, it may be positioned between the inner diameter of the balloon 110 and the outer diameter of the catheter shaft 130. FIG. 2 shows that the distal end 121 may be substantially adjacent to the inner diameter of the balloon 110 and the outer diameter of the catheter shaft 130. Preferably, the proximal-most part of the cutting wire 120 is similarly fed through a proximal aperture corresponding to the distal aperture 196.

Similar to cutting wire 120, the middle portion 124 of cutting wire 125 may be positioned along the working diameter 111 of the balloon 110. Similar to cutting wire 120, the middle portion 124 may also be aligned parallel to the longitudinal axis of the balloon catheter 100. The distal end 123 of the cutting wire 125 may then be positioned such that it extends along the tapered distal portion 112 of the balloon 110 and along the distal neck 113 of the balloon 110. The distal-most part of the distal end 123 may be fed through distal aperture 197. Similar to the distal end 121 of cutting wire 120, the length of the distal end 123 that is fed through distal aperture 197 may vary and is partly dependent upon the region that the balloon catheter 100 is to be deployed within. In the example shown in FIG. 2, the length of the distal end 123 that is fed through distal aperture 197 may range from about 1 mm to about 5 mm.

After the distal-most part of the distal end 123 is fed through distal aperture 196, it may be positioned between the inner diameter of the balloon 110 and the outer diameter of the catheter shaft 130. FIG. 2 shows that the distal end 123 may be substantially adjacent to the inner diameter of the balloon 110 and the outer diameter of the catheter shaft 130. Preferably, the proximal-most part of the cutting wire 125 is similarly fed through a proximal aperture corresponding to the distal aperture 197. The proximal aperture and distal aperture 197 may be longitudinally aligned such that the cutting wire 125 is configured parallel to the longitudinal axis of the balloon catheter 100.

The proximal-most and distal-most ends of the cutting wires 120 and 125 may be surface treated to increase mechanical adhesion of the wires 120, 125 with the surfaces of the balloon 110 and catheter shaft 130. Some metallic materials when used for the wires 120, 125 may not possess enough frictional engagement to lock with the material of the balloon 110 and catheter shaft 130 during heat bonding. For example, if the cutting wires 120 and 125 are formed from a shape memory alloy such as nitinol, the surfaces of the nitinol wires are typically smooth such that they may slip from the bonding site as the material of the balloon 110 and catheter shaft 130 melts and flows around the nitinol surfaces. Accordingly, to compensate for this slippage, the nitinol surfaces may be surface treated to impart surface roughness therealong. The surface roughness of the nitinol wires may create multiple crevices for the melted material of the shaft 130 and balloon 110 to flow thereinto and solidify during heat bonding. Surface treatment may be achieved by any means known to one of ordinary skill in the art, including grit blasting. The ends of the cutting wires 120 and 125 may also be crimped to increase mechanical after bonding.

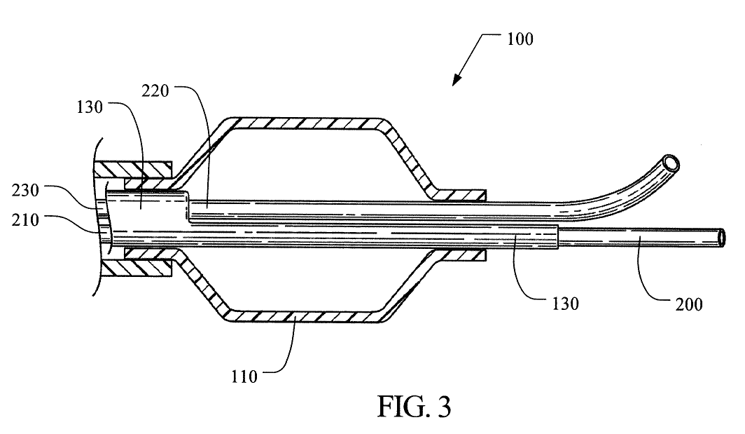

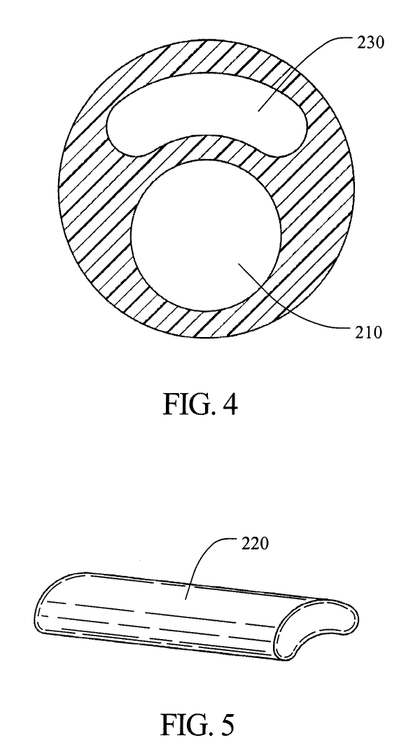

Prior to heat bonding the ends of the cutting wires 120, 125, mandrels may be inserted into the wire guide lumen 210 and inflation lumen 230 of the balloon catheter 100, as shown in FIGS. 2-5. Generally speaking, during the heat bonding process, the wire guide lumen 210 and inflation lumen 230 may have a tendency to collapse. Accordingly, to prevent the collapse of the lumens during heat bonding, FIG. 2 shows that a mandrel 200 may be inserted through the wire guide lumen 210. (For purposes of clarity, FIG. 2 does not show the inflation lumen 230 of the balloon catheter 100). FIG. 3 shows a partial cross-sectional view of the balloon catheter 100 with the mandrel 200 through the wire guide lumen 210 and a second mandrel 220 through the inflation lumen 230. (For purposes of clarity, FIG. 3 does not show the cutting wires disposed in their final configuration before heat bonding). The mandrel 220 may extend pass the distal neck of the balloon, as shown in FIG. 3, to allow it to be removed distally. The mandrel 220 may be removed from the distal end of the balloon catheter 100 before the distal bond is performed. The mandrels 200 and 220 may be solid or, alternatively, any type of a cannula known to one of ordinary skill in the art, including a stainless steel cannula. An example of a mandrel is shown in FIG. 5. FIG. 5 shows an example of a mandrel 220 that may be inserted through the inflation lumen 230 to maintain the lumen 230 open during the heat bonding process. FIG. 4 is a cross-sectional end view of the balloon catheter 100 showing the wire guide lumen 210 and inflation lumen 230 that mandrels 200 and 220 may be inserted through.

After the cutting wires 120, 125 have been configured along the balloon 110 and inserted through their respective apertures at the proximal neck and distal neck 113, and the mandrels 200, 220 have been inserted into the wire guide lumen 210 and inflation lumen 230, respectively, the heat bonding process may begin. The heat bonding process generally involves the application of a sufficient temperature and pressure for a predetermined time to melt the material of the catheter 130 and balloon 110 at their interface, thereby capturing the ends of the wires 120, 125 inside of their respective bonds.

The heat bonding may be accomplished in any way known to one of ordinary skill in the art. One example of heat bonding is shown in FIG. 2. FIG. 2 illustrates a bonding sleeve 195 slidably disposed over the outer surface of the balloon 110. The bonding sleeve 195 may be a heat shrink tubing such as a thin plastic sleeve which may be fitted over the distal balloon neck 113 at the region where the cutting wires 120 and 125 are disposed between the outer diameter of the catheter shaft 130 and the inner diameter of the balloon 110. As FIG. 2 shows, prior to application of heat and pressure, the bonding sleeve 195 has a relatively large diameter and is circumferentially disposed relatively loosely about the distal neck 113 of the balloon 110. Upon heating the bonding sleeve 195, the sleeve 195 reduces in diameter and, in doing so, compresses down over the outer surface of the balloon 110. The bonding sleeve 195 preferably does not melt, and, therefore may be removed after the bonding process. Heat from the bonding sleeve 195 transfers to the catheter shaft 130, distal neck 113 of the balloon 110, and the distal ends 121 and 123 of cutting wires 120, 125. Such heat transfer causes the materials of the balloon distal neck 113, catheter shaft 130 and distal ends 121, 123 of cutting wires 120, 125 to melt. The melting captures the distal ends 121 and 123 of the wires 120, 125 inside of their respective heat bonds. Terminating application of the heat and pressure after a predetermined time at the bond site enables the bonds to cool and solidify. The mandrel 220 may be removed distally after each of the proximal ends of the cutting wires 120, 125 has been bonded to the balloon neck and the shaft 130.

The application of heat to the bonding sleeve 195 may be provided in numerous ways. For example, a laser may be used to heat the bonding sleeve 195. Alternatively, metallic jaws such as copper jaws may be used to heat the bonding sleeve 195. The copper jaws may clamp directly over the bonding sleeve 195. The copper jaws may be heated to apply a substantially uniform heat distribution about the circumference of the region desired to be bonded.

Suitable time, temperature, and pressure parameters for the heat bonding process are dependent on a variety of factors including the types of materials of the catheter shaft 130 and balloon 110 as well as the thickness and durometer of the materials used.

Although a heat bonding process has been described as the means for affixing the cutting wires 20 and 25 to the balloon catheter 100, other types of bonding may be utilized. For example, laser bonding, gluing, or chemical solvent bonding may be used. Additionally, other means for affixing the cutting wires 20 and 25 to the balloon catheter 100 besides bonding may be achieved.

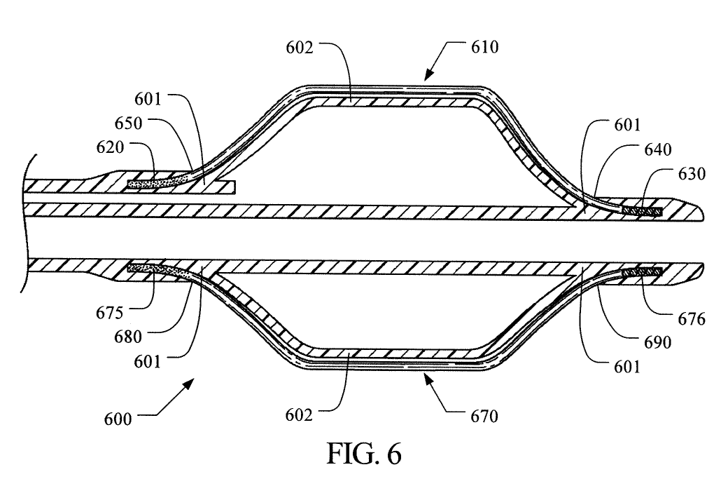

The above-described method of bonding a cutting wire to a surface of an angioplasty balloon may also be applicable to other structural types of dilation elements. For example, FIG. 6 shows a longitudinal cross-sectional view of a balloon catheter 600 having dilation elements 610 and 670. Each of the dilation elements 610 and 670 has two free ends that are bonded under the necks of the balloon 602 and into the material of the shaft 601 and the balloon 602.

One of the free ends of dilation element 610 is shown to be a surface roughened wire 620 that may be fed through a first aperture 650, which is a perforation through the balloon 602 that is sufficiently sized for the surface roughened wire 620 to extend therethrough. The other free end of dilation element 610 is shown to be a coil 630 that may be fed through a second aperture 640, which is a perforation through the balloon 602 that is sufficiently sized for the coil 630 to extend therethrough. First and second apertures 650, 640 may be identical in size or differ in size depending on the outer diameters of surface roughened wire 620 and coil 630. Preferably the first and second apertures 650, 640 are longitudinally aligned with respect to each other.

Similar to dilation element 610, one of the free ends of dilation element 670 is shown to be a surface roughened wire 675 that may be fed through a third aperture 680, which is a perforation through the balloon 602 that is sufficiently sized for the surface roughened wire 675 to extend therethrough. The other free end of dilation element 670 is shown to be a coil 676 that may be fed through a fourth aperture 690 sufficiently sized for the coil 676 to extend therethrough. Third and fourth apertures 680, 690 may be identical in size or differ in size depending on the outer diameters of surface roughened wires 620, 675 and coils 630, 676. Preferably the third and fourth apertures 680, 690 are longitudinally aligned with respect to each other. FIG. 6 shows that dilation element 670 may be circumferentially disposed about 180° from dilation element 610. Other angular separations between the dilations elements 610 and 670 are contemplated. Preferably, the dilation elements 610 and 670 are substantially longitudinally aligned with respect to each other and the longitudinal axis of the balloon catheter 600.

As shown in FIG. 6, each of the surface roughened wires 620 and 675 are embedded into the surfaces of the balloon neck and the catheter shaft 601 to form a bond. The length of the surface roughened wires 620 and 675 that are bonded may range from about 1 mm to about 5 mm. Coils 630 and 676 are also shown embedded into the surfaces of the balloon neck and the catheter shaft 601 at the opposing balloon neck to form a bond. The length of the coils 630 and 676 that are bonded may range from about 1 mm to about 5 mm. The bonds are preferably a heat bond. The bonded regions are preferably the only points of attachment of dilating elements 610 and 670 with the balloon 602. The portions of the dilation elements 610 and 670 extending along the working diameter of the balloon 602 preferably remain unattached therealong. Other types of bonds known to one of ordinary skill in the art are contemplated.

The structural details of the attachment of the dilation elements 610 and 670 to their respective coils is described in the application entitled “Balloon Catheter With Dilating Elements” filed on Feb. 13, 2007, Ser. No. ______ (Attorney Docket No. 8627-1039), which is incorporated herein in its entirety by reference. It should be appreciated that the embodiments disclosed herein may also be applied to other types of dilation elements.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A balloon catheter for dilation of a vessel wall, comprising:
a shaft comprising a distal end and a proximal end;
a balloon mounted on the distal end of the shaft, the balloon comprising a distal neck portion, a proximal neck portion, wherein at least a length of an outer surface of the balloon comprises a working diameter adapted to dilate the vessel wall, the shaft comprising an inflation lumen extending therethrough in fluid communication with an interior region of the balloon, the balloon thereby being expandable between a deflated state and an inflated state; and
a dilation element comprising a proximal end, a distal end, and a middle portion, the middle portion of the dilation element extending along the working diameter of the balloon, the distal end of the dilation element extending through a distal aperture of the balloon at the distal neck portion, the distal end of the dilation element being bonded into the distal neck portion of the balloon and the shaft.

2. The balloon catheter according to claim 1, wherein the proximal end of the dilation element extends through a proximal aperture of the balloon at the proximal neck portion, the proximal end of the dilation element being bonded into the proximal neck portion of the balloon and the shaft.

3. The balloon catheter according to claim 2, wherein the proximal end of the dilation element is heat bonded into the proximal neck portion and the shaft.

4. The balloon catheter according to claim 1, wherein the middle portion of the dilation element comprises a wire.

5. The balloon catheter according to claim 4, wherein at least one of the distal end and the proximal end of the dilation element comprises a coil.

6. The balloon catheter according to claim 1, wherein at least one of the proximal end and the distal end of the dilation element comprises a roughened surface.

7. The balloon catheter according to claim 1, wherein the middle portion of the dilation element is rigid and unattached to the working diameter of the balloon.

8. The balloon catheter according to claim 1, wherein the distal end of the dilation element being bonded into the distal neck portion of the balloon and the shaft has a length between about 1 mm and about 5 mm.

9. The balloon catheter according to claim 1, wherein the distal end of the dilation element is heat bonded into the distal neck portion of the balloon and the shaft.

10. The balloon catheter according to claim 1, wherein the dilation element extends substantially parallel to a longitudinal axis of the shaft.

11. The balloon catheter according to claim 1, wherein a plurality of the dilation elements are circumferentially disposed about the balloon.

12. The balloon catheter according to claim 1, wherein the proximal end of the dilation element extends through a proximal aperture of the balloon at the proximal neck portion, the proximal end of the dilation element being bonded into the proximal neck portion of the balloon and the shaft, wherein the middle portion of the dilation element comprises a wire, wherein at least one of the proximal end and the distal end of the dilation element comprises a roughened surface, wherein the middle portion of the dilation element is rigid and unattached to the working diameter of the balloon, wherein the proximal end of the dilation element is heat bonded into the proximal neck portion and the shaft, and wherein the distal end of the dilation element is heat bonded into the distal neck portion of the balloon and the shaft.

13. A method of bonding a dilation element to a balloon, comprising the steps of:
(a) positioning a dilation element along an outer diameter of a balloon, a middle portion of the dilation element extending along the outer diameter;
(b) forming a proximal aperture along a proximal neck of the balloon;
(c) inserting a proximal end of the dilation element through a proximal aperture located at the proximal neck of the balloon, the proximal end of the dilation element disposed between an inner diameter of the balloon at the proximal neck and an outer diameter of a shaft; and
(d) bonding the proximal end of the dilation element with the balloon and the shaft.

14. The method of claim 13, further comprising the steps of:
(e) forming a distal aperture along a distal neck of the balloon;
(f) inserting a distal end of the dilation element through the distal aperture located at a distal neck of the balloon, the distal end of the dilation element disposed between an inner diameter of the balloon at the distal neck and an outer diameter of the shaft; and
(g) bonding the distal end of the dilation element with the balloon and the shaft.

15. The method of claim 13, wherein step (c) further comprises surface treating the proximal end of the dilation element to increase mechanical adhesion of the dilation element before inserting the proximal end through the proximal aperture.

16. The method of claim 13, wherein step (d) further comprises mounting a bonding sleeve over the proximal neck of the balloon and dilation element.

17. The method of claim 13, wherein step (d) further comprises applying a sufficient amount of heat for a predetermined time to form the heat bond.

18. The method of claim 13 further comprising placing a mandrel within a lumen of the shaft to prevent collapse of the lumen during the heat bonding step.

19. The method of claim 13, wherein the middle portion of the dilation element comprises a wire and the proximal end of the dilation element comprises a coil.

20. The method of claim 14, wherein the middle portion of the dilation element comprises a wire and a distal end of the dilation element comprises a coil.

21. The method of claim 14, wherein step (f) further comprises surface treating the distal end of the dilation element to increase mechanical adhesion of the dilation element before inserting the distal end through the distal aperture.
